(19) United States
(12) Patent Application Publication
(10) Pub. No.: US 2018/0169393 A1
(43) Pub. Date: Jun. 21, 2018

(54) Thoracocentesis Drainage Tube

(71) Applicant: Mei JIANG, Pingxiang City (CN)

(72) Inventor: Mei JIANG, Pingxiang City (CN)

(21) Appl. No.: 15/384,116

(22) Filed: Dec. 19, 2016

(51) Int. Cl. A61M 27/00; A61M 25/00

(52) U.S. Cl. CPC A61M 27/00; A61M 25/0023

(57) ABSTRACT

The invention provides a thoracocentesis drainage tube, comprising a catheter made from a soft elastic material and comprising a main body part and a plurality of partial parts dispersedly distributed on the main body part, wherein the main body part has a wall thickness of 2 mm to 4 mm, and each of the partial parts has a wall thickness of 0.8 mm to 1.5 mm. To patients suffering from pleural effusion for a long time subjected to a great deal of liquid drainage or to patients suffering from pneumothorax, the invention can effectively avoid a problem that the patients suffer from hypoxemia and hypotension in different degrees due to unilateral pneumonedema induced by quick recruitment of lung tissues caused by an excessively high evacuation rate. Furthermore, the invention is simple in structure, easy to manufacture, and convenient for operation.

### FIELD

The invention pertains to the field of medical apparatuses, and more particularly, to a thoracocentesis drainage tube.

### BACKGROUND

At present, in a thoracentesis operation, to patients suffering from pleural effusion for a long time subjected to a great deal of liquid drainage or to patients suffering from pneumothorax, if air evacuation is excessively fast due to lack of experience of operators, the patients suffer from hypoxemia and hypotension in different degrees due to unilateral pneumonedema caused by quick recruitment of lung tissues. Mostly, this case happens immediately or within one hour after lung recruitment, not exceeding 24 hours generally. The patients present as violent coughing, dyspnea, thoracalgia, dysphoria, palpitation and so on, and then present as whooping cough a great deal of white or pink frothy sputum, sometimes accompanied with pyrexia, nausea and emesis, or even shock and coma. Remedy may be performed on the patients by subsequent treatment measures (for example, correcting hypoxemia, stabilizing hemodynamics or providing mechanical ventilation if necessary). However, apparently unnecessary suffering is added to the patients.

### SUMMARY

The invention aims at providing a thoracocentesis drainage tube and is intended to solve the problem that patients suffer from hypoxemia and hypotension in different degrees due to unilateral pneumonedema induced by quick recruitment of lung tissues caused by an excessively high evacuation rate in a thoracentesis operation.

The invention is implemented in this way: a thoracocentesis drainage tube, including a catheter made from a soft elastic material and including a main body part and a plurality of partial parts dispersedly distributed on the main body part, where the main body part has a wall thickness of 2 mm to 4 mm, and each of the partial parts has a wall thickness of 0.8 mm to 1.5 mm.

Preferably, the catheter is made from a soft elastic silica-gel material.

Preferably, every two of the partial parts constitute a group, and every group of partial parts are symmetrically distributed on an opposite direction of a tube wall of the main body part.

Preferably, the main body part has a wall thickness of 3 mm, and each of the partial parts has a wall thickness of 1 mm.

Preferably, the catheter has a pore diameter of 8 mm to 12 mm.

Preferably, each of the partial parts is a circle having a diameter of 8 mm to 14 mm.

Preferably, each of the partial parts is a circle having a diameter of 10 mm

Compared with disadvantages and deficiencies of the prior art, the invention has following beneficial effects: to patients suffering from pleural effusion for a long time subjected to a great deal of liquid drainage or to patients suffering from pneumothorax, the invention can effectively avoid the problem that the patients suffer from hypoxemia and hypotension in different degrees due to unilateral pneumonedema induced by quick recruitment of lung tissues caused by an excessively high evacuation rate. Furthermore, the invention is simple in structure, easy to manufacture, and convenient for operation.

### BRIEF DESCRIPTION OF THE DRAWINGS

The foregoing and other features and advantages of the invention will be apparent from the following description of embodiments thereof as illustrated in the accompanying drawings. The accompanying drawings, which are incorporated herein and form a part of the specification, further serve to explain the principles of the invention and to enable a person skilled in the pertinent art to make and use the invention. The drawings are not to scale.

FIG. 1 is a schematic structural diagram of a thoracocentesis drainage tube according to an embodiment of the invention.

FIG. 2 is a section state diagram when the thoracocentesis drainage tube in FIG. 1 is in use at a lower evacuation rate.

FIG. 3 is a section state diagram when the thoracocentesis drainage tube in FIG. 1 is in use at a lower evacuation rate.

### DETAILED DESCRIPTION OF THE DRAWINGS

Detailed description of the invention will further be made with reference to drawings and embodiments to make the objects, technical solutions and advantages of the invention more apparent. It is to be understood that embodiments described herein are only used to explain the invention, and are not restrictive of the invention.

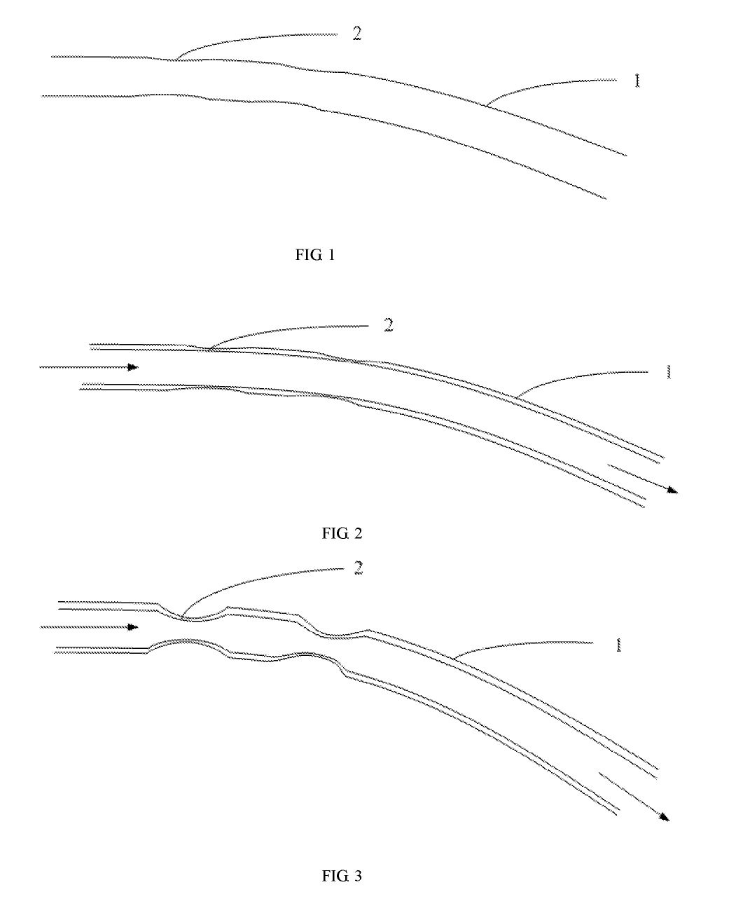

As shown in FIG. 1 to FIG. 3, FIG. 1 is a schematic structural diagram of a thoracocentesis drainage tube according to an embodiment of the invention; FIG. 2 is a section state diagram when the thoracocentesis drainage tube in FIG. 1 is in use at a lower evacuation rate; and FIG. 3 is a section state diagram when the thoracocentesis drainage tube in FIG. 1 is in use at a higher evacuation rate.

A thoracocentesis drainage tube includes a catheter 1 made from a soft elastic material and comprising a main body part and a plurality of partial parts 2 dispersedly distributed on the main body part, where the main body part has a wall thickness of 2 mm to 4 mm, and each of the partial parts 2 has a wall thickness of 0.8 mm to 1.5 mm.

More specifically, in the embodiments of the invention, the catheter has a pore diameter of 8 mm to 12 mm.

In the invention, in a thoracentesis operation, to patients suffering from pleural effusion for a long time subjected to a great deal of liquid drainage or to patients suffering from pneumothorax, if the operator adopt a normal evacuation rate, as shown in FIG. 2, the wall thickness and an elasticity capacity of the partial parts 2 can ensure that the partial parts 2 do not sink toward the tube wall inwardly; and when the evacuation rate is excessively high, as shown in FIG. 3, the partial parts 2 sink, which causes a relative pore diameter of the catheter to lessen. Therefore, a problem of operation failure caused by an excessively high evacuation rate can be effectively avoided.

In practical application of the invention, a relationship among stability of the main body part, sinking of the partial parts 2 and an exhaustion rate depends on multiple factors. In a manufacturing process, a full consideration is taken into the elasticity capacity of the material manufacturing the catheter 1, the wall thicknesses and the areas of the main body part and the partial parts 2 of the catheter 1, and the pore diameter of the catheter 1, etc. In the embodiments of the invention, more specifically, the main body part has a wall thickness of 3 mm, and each of the partial parts has a wall thickness of 1 mm. Each of the partial parts is a circle having a diameter of 8 mm to 14 mm, preferably a circle having a diameter of 10 mm, and the used soft elastic material is a soft elastic silica-gel material frequently used in medical catheter materials.

In a further implementation process, to ensure a better rate-limiting effect, in the embodiments of the invention, every two of the partial parts 2 constitute a group, and every group of partial parts 2 are symmetrically distributed on an opposite direction of a tube wall of the main body part. In an actual application process, setting of the group number of the partial parts 2 also may be determined according to actual situations. For example, an auxiliary catheter made from a harder material may be inserted into the catheter 1, and the group number of the partial parts 2 may be controlled by controlling an insertion depth of the auxiliary catheter. In this way, a problem of controlling an exhaustion rate may be better solved.

To patients suffering from pleural effusion for a long time subjected to a great deal of liquid drainage or to patients suffering from pneumothorax, the invention can effectively avoid a problem that the patients suffer from hypoxemia and hypotension in different degrees due to unilateral pneumonedema induced by quick recruitment of lung tissues caused by an excessively high evacuation rate. Furthermore, the invention is simple in structure, easy to manufacture, and convenient for operation.

The above are merely preferred embodiments of the invention, which are not used to limit the invention. Any variation, identical substitution and improvement made within the spirit and principle of the invention shall fall within the scope of protection of the invention.

What is claimed is:

1. A thoracocentesis drainage tube comprising:
a catheter made from a soft elastic material, the catheter comprising a main body part and a plurality of partial parts dispersedly distributed on the main body part, wherein the main body part has a wall thickness of 2 mm to 4 mm, and each of the plurality of partial parts has a wall thickness of 0.8 mm to 1.5 mm.

2. The thoracocentesis drainage tube according to claim 1, wherein the catheter is made from a soft elastic silica-gel material.

3. The thoracocentesis drainage tube according to claim 2, wherein every two of the plurality of partial parts constitutes a group, and every group of partial parts is symmetrically distributed on opposite direction of a tube wall of the main body part.

4. The thoracocentesis drainage tube according to claim 3, wherein the main body part has a wall thickness of 3 mm, and each of the partial parts has a wall thickness of 1 mm.

5. The thoracocentesis drainage tube according to claim 4, wherein the catheter has a pore diameter of 8 mm to 12 mm.

6. The thoracocentesis drainage tube according to claim 5, wherein each of the partial parts is a circle having a diameter of 8 mm to 14 mm.

7. The thoracocentesis drainage tube according to claim 6, wherein each of the partial parts is a circle having a diameter of 10 mm.
